SEQUENCE LISTING (53 sequences)

SEQ ID NO: 1 - DNA, 18 bp, artificial sequence
  sequence capable of hybridisation with an HPV 16 sequence
gttaccacagttatgcac

SEQ ID NO: 2 - DNA, 18 bp, artificial sequence
  sequence capable of hybridisation with an HPV 16 sequence
gttaccagatttatgcac

SEQ ID NO: 3 - DNA, 18 bp, artificial sequence
  sequence capable of hybridisation with an HPV 16 sequence
gttaccacatttatgcac

SEQ ID NO: 4 - DNA, 16 bp, artificial sequence
  sequence capable of hybridisation with an HPV 16 sequence
gcgacccggaaagtta

SEQ ID NO: 5 - DNA, 21 bp, artificial sequence
  sequence capable of hybridisation with an HPV 16 sequence
ttgttatagtgtgtatggaac

SEQ ID NO: 6 - DNA, 21 bp, artificial sequence
  sequence capable of hybridisation with an HPV 16 sequence
tgttatagtttgtatggaaca

SEQ ID NO: 7 - DNA, 21 bp, artificial sequence
  sequence capable of hybridisation with an HPV 16 sequence
gtgagtatagacattattgtt

SEQ ID NO: 8 - DNA, 23 bp, artificial sequence
  sequence capable of hybridisation with an HPV 16 sequence
tagtgagtatagatattattgtt

SEQ ID NO: 9 - DNA, 18 bp, artificial sequence
  sequence capable of hybridisation with an HPV 16 sequence
ccatatgcagtgtgtgat

SEQ ID NO: 10 - DNA, 18 bp, artificial sequence
  sequence capable of hybridisation with an HPV 16 sequence
ccatatgctgtatgtgat

SEQ ID NO: 11 - DNA, 23 bp, artificial sequence
  sequence capable of hybridisation with an HPV 16 sequence
tattatctcatgtatagttgtgg

SEQ ID NO: 12 - DNA, 17 bp, artificial sequence
  sequence capable of hybridisation with an HPV 16 sequence
gcgacccacaaagttac

SEQ ID NO: 13 - DNA, 19 bp, artificial sequence
  sequence capable of hybridisation with an HPV 16 sequence
tgtgatttgttgattaggt

SEQ ID NO: 14 - DNA, 18 bp, artificial sequence
  sequence capable of hybridisation with an HPV 16 sequence
tgttctcgatgatctgca

SEQ ID NO: 15 - DNA, 19 bp, artificial sequence
  sequence capable of hybridisation with an HPV 16 sequence
agcagacattttatgcacc

SEQ ID NO: 16 - DNA, 19 bp, artificial sequence
  sequence capable of hybridisation with an HPV 16 sequence
gctcataacagtagagatc

SEQ ID NO: 17 - DNA, 20 bp, artificial sequence
  sequence capable of hybridisation with an HPV 16 sequence
tgtttcaggacccacaggag

SEQ ID NO: 18 - DNA, 21 bp, artificial sequence
  sequence capable of hybridisation with an HPV 16 sequence
tagttgtttgcagctctgtgc

SEQ ID NO: 19 - DNA, 21 bp, artificial sequence
  sequence capable of hybridisation with an HPV 16 sequence
ttgcttgcagtacacacattc

SEQ ID NO: 20 - DNA, 19 bp, artificial sequence
  sequence capable of hybridisation with an HPV 16 sequence
gcgacccrbaaagttacca

SEQ ID NO: 21 - DNA, 21 bp, artificial sequence
  sequence capable of hybridisation with an HPV 16 sequence
tagagatgggaatccatatgc

SEQ ID NO: 22 - DNA, 22 bp, artificial sequence
  sequence capable of hybridisation with an HPV 16 sequence
ctgttctaatgttgttccatac

SEQ ID NO: 23 - DNA, 27 bp, artificial sequence
  sequence capable of hybridisation with an HPV 16 sequence
tagtgagtatagayattattgttatag

SEQ ID NO: 24 - DNA, 21 bp, artificial sequence
  sequence capable of hybridisation with an HPV 16 sequence
gcaatacaacaaaccgttgtg

SEQ ID NO: 25 - DNA, 20 bp, artificial sequence
  sequence capable of hybridisation with an HPV 16 sequence
gacacagtggcttttgacag

SEQ ID NO: 26 - DNA, 24 bp, artificial sequence
  sequence capable of hybridisation with an HPV 16 sequence
tgtgatttgttrattaggtgtatt

SEQ ID NO: 27 - DNA, 21 bp, artificial sequence
  sequence capable of hybridisation with an HPV 16 sequence
tcgatgtatgtcttgttgcag

SEQ ID NO: 28 - DNA, 21 bp, artificial sequence
  sequence capable of hybridisation with an HPV 16 sequence
atctccatgcatgattacagc

SEQ ID NO: 29 - DNA, 21 bp, artificial sequence
  sequence capable of hybridisation with an HPV 16 sequence
ctgggtttctctacgtgttct

SEQ ID NO: 30 - DNA, 22 bp, artificial sequence
  sequence capable of hybridisation with an HPV 16 sequence
agtgagtatagatattattgtt

SEQ ID NO: 31 - DNA, 7904 bp, hpv 16 sequence/genbank K02718.1
  sequence capable of hybridisation with an HPV 16 sequence
actacaataattcatgtataaaactaagggcgtaaccgaaatcggttgaaccgaaaccggttagtataaaagcagacattttatgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataatctaccatggctgatcctgcaggtaccaatggggaagagggtacgggatgtaatggatggttttatgtagaggctgtagtggaaaaaaaaacaggggatgctatatcagatgacgagaacgaaaatgacagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatttggtagtccacttagtgatattagtggatgtgtagacaataatattagtcctagattaaaagctatatgtatagaaaaacaaagtagagctgcaaaaaggagattatttgaaagcgaagacagcgggtatggcaatactgaagtggaaactcagcagatgttacaggtagaagggcgccatgagactgaaacaccatgtagtcagtatagtggtggaagtgggggtggttgcagtcagtacagtagtggaagtgggggagagggtgttagtgaaagacacactatatgccaaacaccacttacaaatattttaaatgtactaaaaactagtaatgcaaaggcagcaatgttagcaaaatttaaagagttatacggggtgagtttttcagaattagtaagaccatttaaaagtaataaatcaacgtgttgcgattggtgtattgctgcatttggacttacacccagtatagctgacagtataaaaacactattacaacaatattgtttatatttacacattcaaagtttagcatgttcatggggaatggttgtgttactattagtaagatataaatgtggaaaaaatagagaaacaattgaaaaattgctgtctaaactattatgtgtgtctccaatgtgtatgatgatagagcctccaaaattgcgtagtacagcagcagcattatattggtataaaacaggtatatcaaatattagtgaagtgtatggagacacgccagaatggatacaaagacaaacagtattacaacatagttttaatgattgtacatttgaattatcacagatggtacaatgggcctacgataatgacatagtagacgatagtgaaattgcatataaatatgcacaattggcagacactaatagtaatgcaagtgcctttctaaaaagtaattcacaggcaaaaattgtaaaggattgtgcaacaatgtgtagacattataaacgagcagaaaaaaaacaaatgagtatgagtcaatggataaaatatagatgtgatagggtagatgatggaggtgattggaagcaaattgttatgtttttaaggtatcaaggtgtagagtttatgtcatttttaactgcattaaaaagatttttgcaaggcatacctaaaaaaaattgcatattactatatggtgcagctaacacaggtaaatcattatttggtatgagtttaatgaaatttctgcaagggtctgtaatatgttttgtaaattctaaaagccatttttggttacaaccattagcagatgccaaaataggtatgttagatgatgctacagtgccctgttggaactacatagatgacaatttaagaaatgcattggatggaaatttagtttctatggatgtaaagcatagaccattggtacaactaaaatgccctccattattaattacatctaacattaatgctggtacagattctaggtggccttatttacataatagattggtggtgtttacatttcctaatgagtttccatttgacgaaaacggaaatccagtgtatgagcttaatgataagaactggaaatcctttttctcaaggacgtggtccagattaagtttgcacgaggacgaggacaaggaaaacgatggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaagtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccaccccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatgacaaatcttgatactgcatccacaacattactggcgtgctttttgctttgctttgtgtgcttttgtgtgtctgcctattaatacgtccgctgcttttgtctgtgtctacatacacatcattaataatattggtattactattgtggataacagcagcctctgcgtttaggtgttttattgtatatattatatttgtttatataccattatttttaatacatacacatgcacgctttttaattacataatgtatatgtacataatgtaattgttacatataattgttgtataccataacttactattttttcttttttattttcatatataatttttttttttgtttgtttgtttgttttttaataaactgttattacttaacaatgcgacacaaacgttctgcaaaacgcacaaaacgtgcatcggctacccaactttataaaacatgcaaacaggcaggtacatgtccacctgacattatacctaaggttgaaggcaaaactattgctgaacaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttctgatccttctatagtttctttagtggaagaaactagttttattgatgctggtgcaccaacatctgtaccttccattcccccagatgtatcaggatttagtattactacttcaactgataccacacctgctatattagatattaataatactgttactactgttactacacataataatcccactttcactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaggttaaagttgtagaccctgcttttgtaaccactcccactaaacttattacatatgataatcctgcatatgaaggtatagatgtggataatacattatatttttctagtaatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgatttaagtactattgatcctgcagaagaaatagaattacaaactataacaccttctacatatactaccacttcacatgcagcctcacctacttctattaataatggattatatgatatttatgcagatgactttattacagatacttctacaaccccggtaccatctgtaccctctacatctttatcaggttatattcctgcaaatacaacaattccttttggtggtgcatacaatattcctttagtatcaggtcctgatatacccattaatataactgaccaagctccttcattaattcctatagttccagggtctccacaatatacaattattgctgatgcaggtgacttttatttacatcctagttattacatgttacgaaaacgacgtaaacgtttaccatattttttttcagatgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttcatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtactgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacccaggcaattgcttgtcaaaaacatacacctccagcacctaaagaagatgatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaagtattgtatgtatgttgaattagtgttgtttgttgtgtatatgtttgtatgtgcttgtatgtgcttgtaaatattaagttgtatgtgtgtttgtatgtatggtataataaacacgtgtgtatgtgtttttaaatgcttgtgtaactattgtgtcatgcaacataaataaacttattgtttcaacacctactaattgtgttgtggttattcattgtatataaactatatttgctacatcctgtttttgttttatatatactatattttgtagcgccaggcccattttgtagcttcaaccgaattcggttgcatgctttttggcacaaaatgtgtttttttaaatagttctatgtcagcaactatggtttaaacttgtacgtttcctgcttgccatgcgtgccaaatccctgttttcctgacctgcactgcttgccaaccattccattgttttttacactgcactatgtgcaactactgaatcactatgtacattgtgtcatataaaataaatcactatgcgccaacgccttacataccgctgttaggcacatatttttggcttgttttaactaacctaattgcatatttggcataaggtttaaacttctaaggccaactaaatgtcaccctagttcatacatgaactgtgtaaaggttagtcatacattgttcatttgtaaaactgcacatgggtgtgtgcaaaccgattttgggttacacatttacaagcaacttatataataatactaa

SEQ ID NO: 32 - DNA, 456 bp, HPV 16 E6 from deposit AF402678_AA
  sequence capable of hybridisation with an HPV 16 sequence
atgtttcaggacccacaggagcgacccagaaagttaccacatttatgcacagagctgcaaacaactatacatgatataatattagaatgtgtgtactgcaagcaacagttactgcgacgtgaggtatatgactttgcttttcgggatttatgcatagtatatagagatgggaatccatatgcagtgtgtgataaatgtttaaagttttattctaaaattagtgagtatagatattattgttatagtgtgtatggaacaacattagaacagcaatacaacaaaccgttgtgtgatttgttaattaggtgtattaactgtcaaaagccactatgtcctgaagaaaagcaaagacatctggacaaaaagcaaagattccataatataaggggtcggtggaccggtcgatgtatgtcttgttgcagatcatcgagaacacgtagagaaacccagctgtaa

SEQ ID NO: 33 - DNA, 456 bp, HPV 16 E6 from deposit AF472508_AF1
  sequence capable of hybridisation with an HPV 16 sequence
atgtttcaggacccacaggagcgacccacaaagttaccagatttatgcacagagctgcaaacaactatacatgatataatattagaatgtgtgtactgcaagcaacagttactgcgacgtgaggtatatgactttgcttttcgggatttatgcatagtatatagagatgggaatccatatgcagtgtgtgataaatgtttaaagttttattctaaaattagtgagtatagatattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 34 - DNA, 456 bp, HPV 16 E6 from deposit AF472509_AF2
  sequence capable of hybridisation with an HPV 16 sequence
atgttccaggacccacaggagcgacccataaagttaccagatttatgcacagagctgcaaacaactatacatgatataatattagaatgtgtgtactgcaagcaacagttactgcgacgtgaggtatatgactttgcttttcgggatttatgcatagtatatagagatgggaatccatatgcagtgtgtgataaatgtttaaagttttattctaaaattagtgagtatagatattattgttatagtttgtatggaacaacattagaacagcaatacaacaaaccgttgtgtgatttgttgattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 35 - DNA, 456 bp, HPV 16 E6 from deposit AF486322_E109C
  sequence capable of hybridisation with an HPV 16 sequence
atgttccaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtgtgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 36 - DNA, 456 bp, HPV 16 E6 from deposit AF486325_NA
  sequence capable of hybridisation with an HPV 16 sequence
atgtttcaggacccacaggagcgacccagaaagttaccacatttatgcacagagctgcaaacaactatacatgatataatattagaatgtgtgtactgcaagcaacagttactgcgacgtgaggtatatgactttgcttttcgggatttatgcatagtatatagagatgggaatccatatgcagtgtgtgataaatgtttaaagttttattctaaaattagtgagtatagatattattgttatagtgtgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 37 - DNA, 456 bp, HPV 16 E6 from deposit AF534061_EA
  sequence capable of hybridisation with an HPV 16 sequence
atgtttcaggacccacaggagcgacccagaaagttaccacagttatgcacagagctgcaaacaactatacatgagataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 38 - DNA, 456 bp, HPV 16 E6 from deposit AF536179_E
  sequence capable of hybridisation with an HPV 16 sequence
atgtttcaggacccacaggagcgacccggaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtgtgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 39 - DNA, 456 bp, HPV 16 E6 from deposit AF536180_AF1; identical to SEQ ID NO: 33
  sequence capable of hybridisation with an HPV 16 sequence

SEQ ID NO: 40 - DNA, 456 bp, HPV 16 E6 from deposit AJ388063_AF2; identical to SEQ ID NO: 34
  sequence capable of hybridisation with an HPV 16 sequence

SEQ ID NO: 41 - DNA, 456 bp, HPV 16 E6 from deposit AY098918_AF2; identical to SEQ ID NO: 34
  sequence capable of hybridisation with an HPV 16 sequence

SEQ ID NO: 42 - DNA, 456 bp, HPV 16 E6 from deposit AY098922_AF1; identical to SEQ ID NO: 33
  sequence capable of hybridisation with an HPV 16 sequence

SEQ ID NO: 43 - DNA, 456 bp, HPV 16 E6 from deposit AY098923_AA
  sequence capable of hybridisation with an HPV 16 sequence
atgtttcaggacccacaggagcgacccagaaagttaccacatttatgcacagagctgcaaacaactatacatgatataatattagaatgtgtgtactgcaagcaacagttactgcgacgtgaggtatatgactttgcttttcgggatttatgcatagtatatagagatgggaatccatatgcagtgtgtgataaatgtttaaagttttattctaaaattagtgagtatagatattattgttatagtgtgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcgagaacacgtagagaaacccagctgtaa

SEQ ID NO: 44 - DNA, 456 bp, HPV 16 E6 from deposit HPA388066E350T
  sequence capable of hybridisation with an HPV 16 sequence
atgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 45 - DNA, 456 bp, HPV 16 E6 from deposit K02718_E; identical to SEQ ID NO: 44
  sequence capable of hybridisation with an HPV 16 sequence

SEQ ID NO: 46 - DNA, 456 bp, HPV 16 E6 from deposit U34112_AA; identical to SEQ ID NO: 43
  sequence capable of hybridisation with an HPV 16 sequence

SEQ ID NO: 47 - DNA, 456 bp, HPV 16 E6 from deposit U34116NA; identical to SEQ ID NO: 36
  sequence capable of hybridisation with an HPV 16 sequence

SEQ ID NO: 48 - DNA, 456 bp, HPV 16 E6 from deposit U34121E350T; identical to SEQ ID NO: 44
  sequence capable of hybridisation with an HPV 16 sequence

SEQ ID NO: 49 - DNA, 456 bp, HPV 16 E6 from deposit U34126E109C
  sequence capable of hybridisation with an HPV 16 sequence
atgttccaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 50 - DNA, 456 bp, HPV 16 E6 from deposit U34129EA; identical to SEQ ID NO: 37
  sequence capable of hybridisation with an HPV 16 sequence

SEQ ID NO: 51 - DNA, 456 bp, HPV 16 E6 from deposit U34133_E109C; identical to SEQ ID NO: 35
  sequence capable of hybridisation with an HPV 16 sequence

SEQ ID NO: 52 - DNA, 456 bp, HPV 16 E6 from deposit U34135_E131G; identical to SEQ ID NO: 38
  sequence capable of hybridisation with an HPV 16 sequence

SEQ ID NO: 53 - DNA, 456 bp, HPV 16 E6 from deposit U89348_E131G; identical to SEQ ID NO: 38
  sequence capable of hybridisation with an HPV 16 sequence

(19) United States
(12) Patent Application Publication
(10) Pub. No.: US 2009/0162843 A1
(43) Pub. Date: Jun. 25, 2009

(54) IDENTIFICATION OF HPV16 LINEAGE GROUP

(76) Inventors: Margretha Klazina Kamp, Voorburg (NL); Gijsbertus Everardus Maria Kleter, Voorburg (NL); Wilhelmus Gregorius Quint, Voorburg (NL); Leendert Jan Van Doorn, Voorburg (NL)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/814,207

(22) PCT Filed: Jan. 17, 2006
(86) PCT No.: PCT/EP2006/000417

§ 371 Date: Aug. 6, 2008

(30) Foreign Application Priority Data
Jan. 18, 2005 (GB) 0500999.8

(51) Int. Cl. C12Q 1/68

(52) U.S. Cl. 435/6

(57) ABSTRACT

A method for identification of an HPV16 lineage group in a sample, comprising contacting such nucleic acid simultaneously with three probes, each probe being capable of specific hybridization across positions 143 and 145 of a HPV 16 genome.

### FIELD OF THE INVENTION

The present invention relates to the field of detection and identification of Human Papillomavirus (HPV) infections

### BACKGROUND OF THE INVENTION

Cervical cancer is the second most common malignancy in women, following breast cancer. Carcinoma of the cervix is unique in that it is the first major solid tumor in which HPV DNA is found in virtually all cases and in precursor lesions worldwide.

Over 100 HPV types have been characterized and are numbered in chronological order of isolation. HPV is epitheliotropic and infects only the skin (cutaneous types) or the mucosa of the respiratory and anogenital tract (mucosal types). More than 40 HPV types are known to infect the uterine cervix. Based on the induced benign, premalignant or malignant lesions, HPV is divided into low-risk (e.g., HPV types 6, 11, 42, 43 and 44) and high-risk types (e.g., types 16, 18, 31, 33 and 45), respectively. The high-risk types account for more than 99% of all invasive cervical cancers. Consequently, detection and identification of HPV types is very important. The high-risk types are by definition consistently found in high grade SIL (Squamous Intraepithelial Lesion) and carcinoma in-situ whereas low risk types are mainly found in low grade SIL. This epidemiological observation is supported by molecular findings. For instance, the E6 and E7 proteins from low-risk types 6 and 11 bind p53 and pRB too weakly to immortalize keratinocytes in vitro or to induce malignant transformation in vivo (Woodworth et al., 1990). The circular ds-DNA genome of low-risk HPV types remains episomal whereas the genome of high-risk HPV types is able to integrate into the human genome.

Screening for malignant and premalignant disorders of the cervix is usually performed according to the Papanicoloau (PAP) system. The cervical smears are examined by light microscopy and the specimens containing morphologically abnormal cells are classified into PAP I to V, at a scale of increasing severity of the lesion. This cytomorphological method is an indirect method and measures the possible outcome of an HPV infection. Therefore, HPV DNA detection and typing is of importance in secondary screening in order to select patients for monitoring (follow-up) and treatment. This means that cervical smears classified as PAP II (atypical squamous metaplasia) or higher classes should be analyzed for low-risk and high risk HPV types. Follow-up studies have shown that only high-risk HPV types are involved in the progression from cytologically normal cervix cells to high grade SIL (Remminck et al., 1995). These results indicate that the presence of high-risk HPV types is a prognostic marker for development and detection of cervical cancer.

Diagnosis of HPV by culture is not possible. Also diagnosis by detection of HPV antibodies appears to be hampered by insufficient sensitivity and specificity. Direct methods to diagnose an HPV infection are mainly based on detection of the viral DNA genome by different formats of DNA/DNA or RNA/DNA hybridization with or without prior amplification of HPV DNA. The polymerase chain reaction (PCR) is a method that is highly efficient for amplification of minute amounts of target DNA. Nowadays, mainly three different primer pairs are used for universal amplification of HPV DNA (“broad spectrum primers”). These three primer pairs, MY11/MY09, GP5/GP6 and the SPF10 system, are directed to conserved regions among different HPV types in the LI region (Manos et al., 1989; Van den Brule et al., 1990, WO9914377). The PGMY system, a modification of the MY09/11 is also used (see Gravitt, P., 2000. Improved amplification of genital human papillomaviruses. J. Clin. Microbiol. 38:357-361). Another primer pair, CPI/CPIIg, is directed to conserved regions in the E1 region (Tieben et al., 1993) but CPI/II is not often used.

There are several methods to identify the various HPV types.

HPV DNA can be typed by PCR primers that recognize only one specific type. This method is known as type-specific PCR. Such methods have been described for HPV types 6, 11, 16, 18, 31 and 33 (Claas et al., 1989; Cornelissen et al., 1989; Falcinelli et al., 1992; Van den Brule et al., 1990; Young et al., 1989). The primers are aimed at the E5, L1, E6, L1, E2 and E1 regions of the BPV genome for types 6, 11, 16, 18, 31 and 33, respectively (Baay et al., 1996).

Another method is general amplification of a genomic part from all HPV types followed by hybridization with two cocktails of type-specific probes differentiating between the oncogenic and non-oncogenic groups, respectively. A similar typing method has been described without prior amplification of HPV DNA. In the hybrid capture assay (Hybrid Capture Sharp Assay; Digene, Silver Springs, Md.), each sample is tested for a group of “high-risk” HPV types (eg 16, 18, 31, 33, 35, 39, 45, 51, 52, 56, 58, 59 and 68) and for another group of “low-risk” HPV types (eg 6, 11, 42, 43 and 44) (Cox et al., 1995).

A detection and typing system disclosed in WO9914377, utilises a PCR amplification step and a reverse line blot hybridization with type specific probes.

At present, formal classification of human papillomaviruses is based on sequence analysis of a 291 bp fragment from the L1 region (Chan et al. J Virol. 1995 May; 69(5):3074-83, DeVilliers et al., Virology. 2004 Jun. 20; 324(1):17-27). Phylogenetic analysis of these sequences allows classification of the different HPV types. By definition, if the sequence difference across this region between two HPV isolates is higher than 10% they are classified as different HPV types. Consequently, if the sequence differs more than 10% from any known HPV type it is classified as a novel BPV type. HPV isolates that differ between 2-10% are classified as different subtypes. Finally, if the sequence variation is below 2%, the 2 isolates are classified within the same subtype as different variants.

The biological relevance of HPV subtypes, such as HPV 16 subtypes, in the development and/or progression of disease is not well understood. However, some groups have suggested links between different HPV subtypes and aspects of disease—for example, a predilection of adenocarcinoma for non European variants of HPV 16 (Burk et al Cancer research 63, 7215-7720, 2003). The present invention relates to methods and tools which allow subtypes and/or variants of given HPV types to be analysed.

### STATEMENT OF INVENTION

The present invention relates to a method for the subtyping of any HPV 16 nucleic acid possibly present in a sample, the method comprising the steps of contacting any such nucleic acid with a probe capable of specific hybridization to the HPV 16 genome across a region including position 143 of the HPV 16 genome, and a probe capable of specific hybridization to the HPV 16 genome across a region including position 145 of the HPV 16 genome, said positions given with reference to the sequence of FIG. 1, wherein the hybridization or absence of hybridization gives information as to the nucleotide at position 143 and 145 of the HPV 16 genome.

The invention further relates to a method in which an amplification step is carried out to amplify any HPV 16 nucleic acid possibly present in a biological sample prior to the hybridization step.

The invention further relates to a method in which an amplification step is carried out to amplify any signal used to detect hybridisation of the probe with any HPV nucleic acid possibly present in a biological sample, suitably after amplification of any target nucleic acid.

The invention further relates to oligonucleotide probes and primers enabling said method of detection and/or identification, including subtyping, of HPV.

The invention further relates to protocols according to which said amplification and hybridization steps can be performed. One format for the hybridization step is, for instance, the reverse hybridization format, and more particularly a line probe assay such as the LIPA technique.

The invention further relates to kits comprising primers and/or probes and/or instructions for use in carrying out the invention.

In a further aspect, the invention relates to probes that are capable of being used under the same hybridisation conditions to identify subtypes of HPV 16.

### FIGURES

FIG. 1 gives the sequence of HPV 16 from the Los Alamos strain Genbank accession number K02718.1;

FIG. 2 gives the variation in sequence between different HPV 16 subtypes at defined positions;

FIG. 3 gives the phylogenetic tree generated across the region in FIG. 6;

FIG. 4 gives the HPV 16 probe reactivities;

FIG. 5 gives results that might be obtained using the primers of Table 3;

FIG. 6 gives sequences of HPV 16 E6 variants; and

FIG. 7 indicates mutations found in the HPV 16E6 region.

### DETAILED DESCRIPTION

The present invention relates to a method for the subtyping of any HPV 16 nucleic acid possibly present in a sample, the method generally comprising identification of the nucleotides at positions 143 and 145 of HPV 16 using probes which specifically hybridise across these regions (i.e. to regions of HPV 16 comprising the nucleotides at these positions) and which allow the nucleotides at these positions to be identified.

This invention allows the identification of the lineage group of HPV 16—either European/Asian, American Asian/N. American or African.

The method comprises the steps of contacting such nucleic acid with a probe capable of specific hybridization to the HPV 16 genome across a region including position 143 of the HPV 16 genome, and a probe capable of specific hybridization to the HPV 16 genome across a region including position 145 of the HPV 16 genome, positions given with reference to the sequence of FIG. 1, wherein the hybridization or absence of hybridization of a probe at these positions gives information as to the nucleotide at position 143 and 145 of the HPV 16 genome.

The hybridisation of probe with target nucleic acid takes place under reaction conditions where specific hybridisation of the probe can occur.

We have determined that the identity of nucleotides at these positions in HPV 16 allows the HPV 16 subtype to be generally classed as European/Asian, American Asian/N. American or African lineage type. Given the suggested predilection of adenocarcinoma for non European variants of HPV 16, (Burk et al supra) such a general classification can assist with analysis of HPV disease.

More especially, the different lineage types have the following characteristic nucleotides at positions 143 and 145:

TABLE 1

| | Position 143 | Position 145 |
|---|---|---|
| European/Asian | C | G |
| Asian- | C | T |
| American/N | | |
| american | | |
| African | G | T |

Moreover it has been determined that the region 71-640 of HPV 16 shown underlined in FIG. 1 is particularly informative in respect of HPV subtyping. Probes used in the invention to identify nucleotides at positions 143 and 145 can be used in combination with other probes with the region 71-640, described herein, to provide a more exact picture of the HPV subtype.

The present invention also relates to an isolated nucleic acid sequence consisting of the region 71-640 of HPV 16.

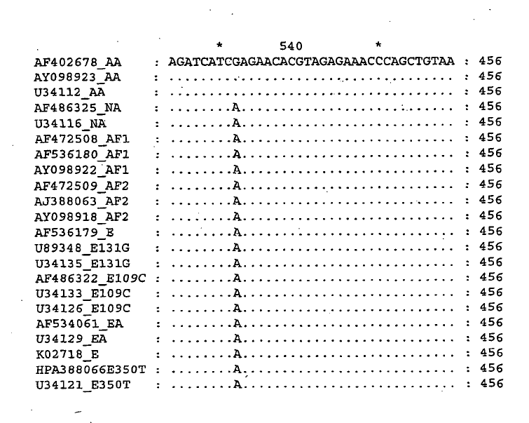

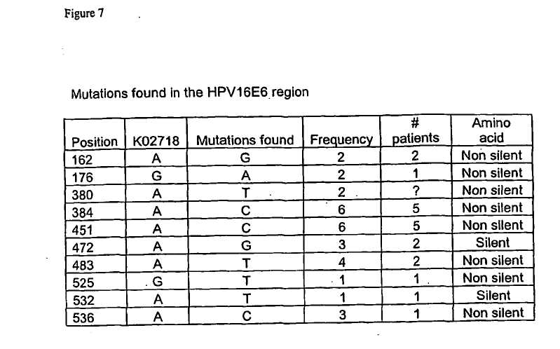

Moreover a number of sequences and mutations have been identified that are not found in the Genbank database. These mutations are listed in FIG. 7. HPV 16 nucleic acids, such as whole genomes or fragments thereof, comprising mutations at these positions are claimed herein. Thus the invention relates to a nucleic acid of HPV 16, or fragment thereof, comprising a mutation as indicated in FIG. 7 (positioning with respect to the K02718 genome). Nucleic acid fragments may be 15, 20, 25, 30, 40, 50, 75, 100, 200 or more nucleotides in length.

### Region of HPV16

Preferably the specific probes used in the invention to subtype HPV 16 are capable of specific hybridisation to HPV 16 nucleic acid across a region including position 143 and/or 145 of the HPV 16 genome, where this numbering is given by reference to the Los Alamos reference sequence (FIG. 1). It will be appreciated that reference to this region using the numbering of the Los Alamos sequence herein includes equivalent regions of other HPV 16 sequences (which may vary from the Los Alamos reference strain), where the equivalent region is identified on the basis of, for example, sequence homology or identity with the sequence of FIG. 1.

Sequence comparisons of nucleic acid identity/homology are readily carried out by the skilled person, for example using the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the default parameters, to determine percent sequence identity for the polynucleotides of the invention. Software for performing BLAST analyses is publicly available through the National Center for Biotechnology Information.

Thus the invention can be seen to relate to probes and to the use of probes which are capable of specific hybridization across a region including position 143 and/or 145 of the HPV 16 genome, said regions being indicated in FIG. 1 or are capable of specific hybridization within an equivalent region in another HPV 16 genome, suitably as assessed by nucleic acid identity or homology.

### Probes

Probes which specifically hybridise across positions 143 and/or 145 of the HPV 16 genome are able to provide information as to the subtype of the HPV strain, either alone or in combination with information from another probe or probes.

Probes for use in the present invention suitably hybridise across both positions 143 and 145.

In one aspect of the invention the analysis of subtype is at the level of phylogenetic based lineage group—namely European/Asian, American Asia. American or African.

In a further aspect of the invention the analysis of subtype is at the level of variants of these lineage groups—eg African 1 and African 2.

“Subtyping”, of HPV 16, as used herein, thus can refer to identification of lineage groups or lineage groups variants, unless otherwise apparent from the context, and the term “HPV subtype” can mean lineage group or lineage group variant.

According to one preferred embodiment of the present invention, a probe is capable of specific hybridization with the genome of only one HPV 16 subtype lineage group or HPV 16 lineage group variant, and thus enables specific identification of this HPV16 subtype when this type is present in a biological sample.

Probes that give information about HPV16 subtype lineage groups or subtype variants are generally considered as subtype specific probes herein and preferred subtype specific probes are capable of specific hybridization across a region including position 143 and/or 145 of the HPV 16 genome of only one HPV16 subtype.

In one aspect the invention relates to the simultaneous use of 3 probes in HPV 16 subtyping, (probe 1, probe 2 and probe 3), each probe capable of specific hybridization across positions 143 and 145 of the HPV 16 genome, the probes comprising the following nucleotide at position 143 and 145:

TABLE 2

| | 143 | 145 |
|---|---|---|
| Probe 1 | C | G |
| Probe 2 | G | T |
| Probe 3 | C | T |

Preferred probes for use in identification of the nucleotides at position 143/145 of IPV 16 nucleic acid are (5′-3):

| | |
|---|---|
| Seq ID no 1: | gttaccaCaGttatgcac |
| Seq ID no 2: | gttaccaGaTttatgcac |
| Seq ID no 3: | gttaccaCaTttatgcac |

These probes are claimed individually and in combination.

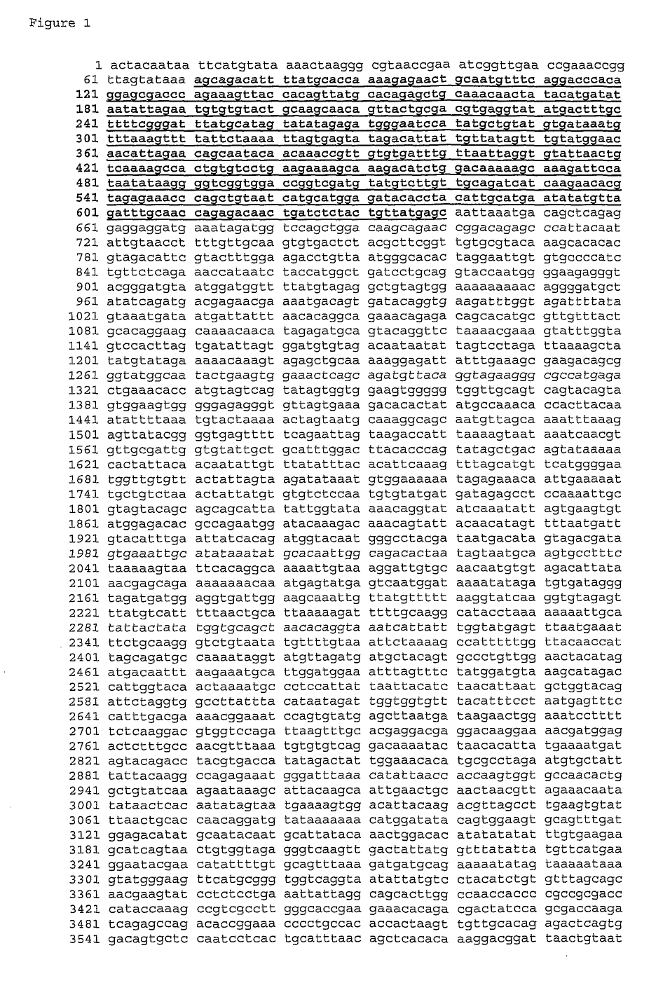

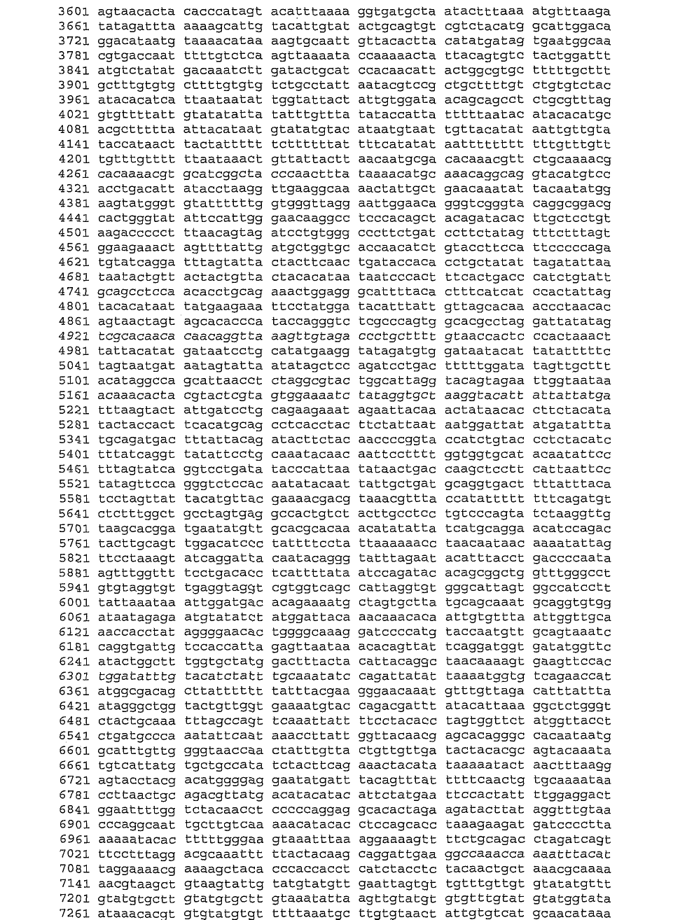

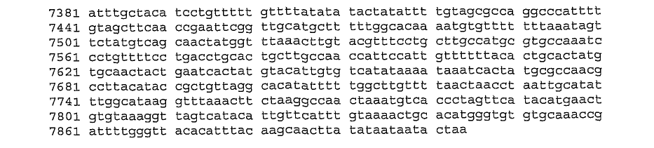

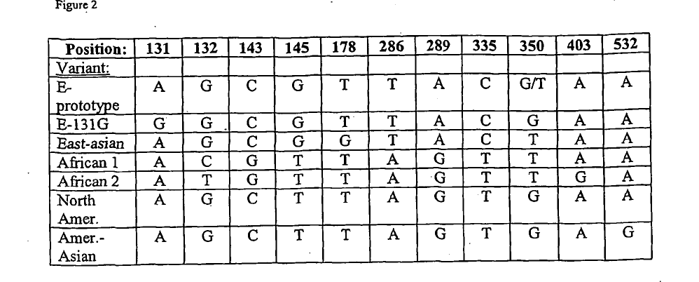

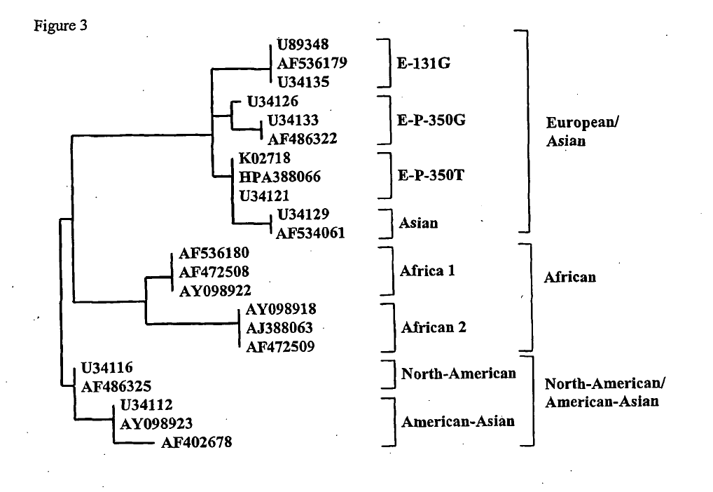

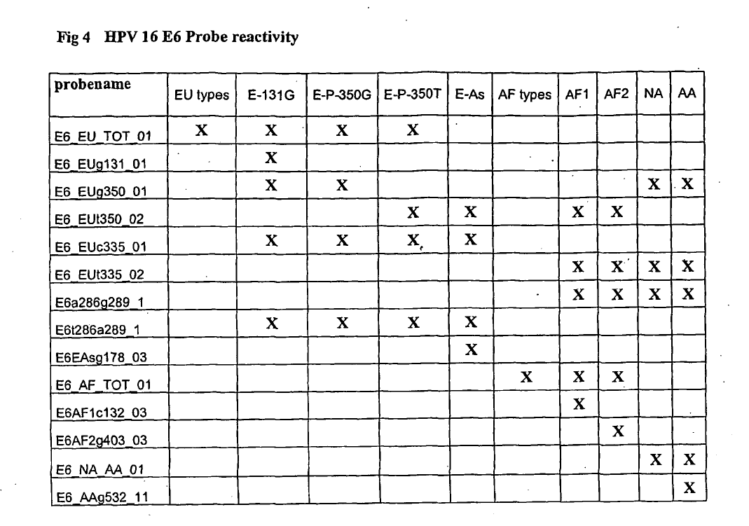

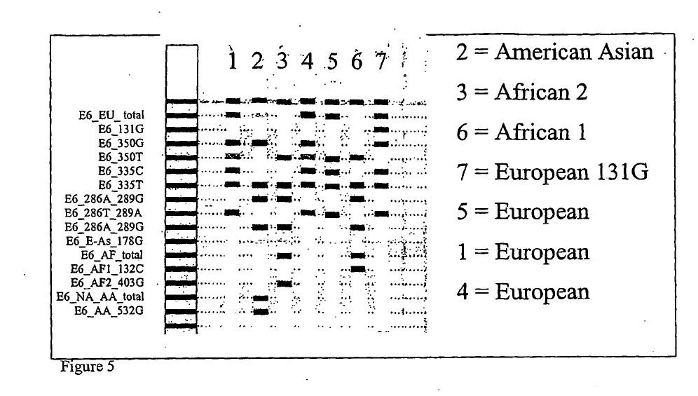

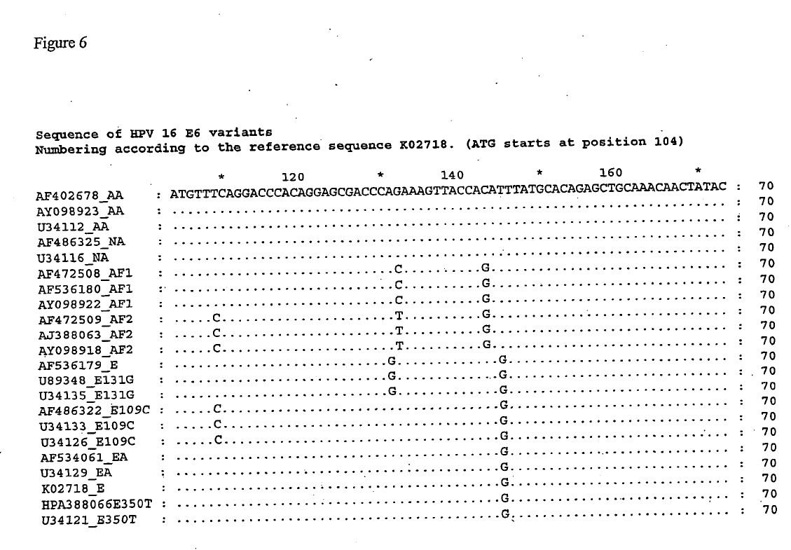

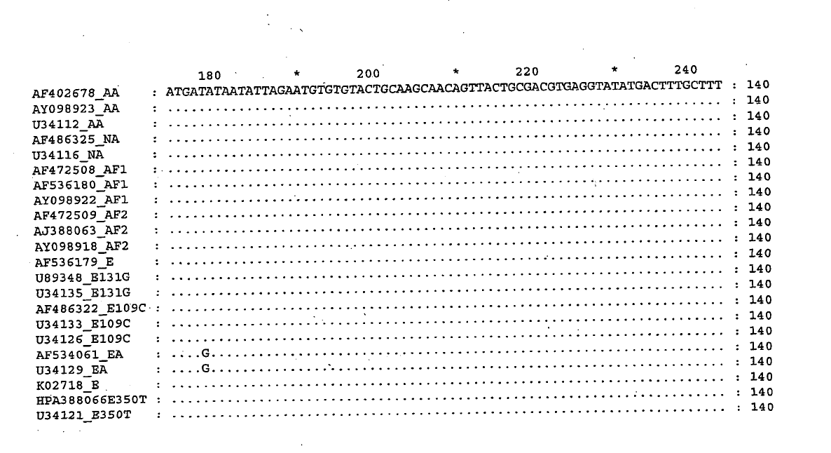

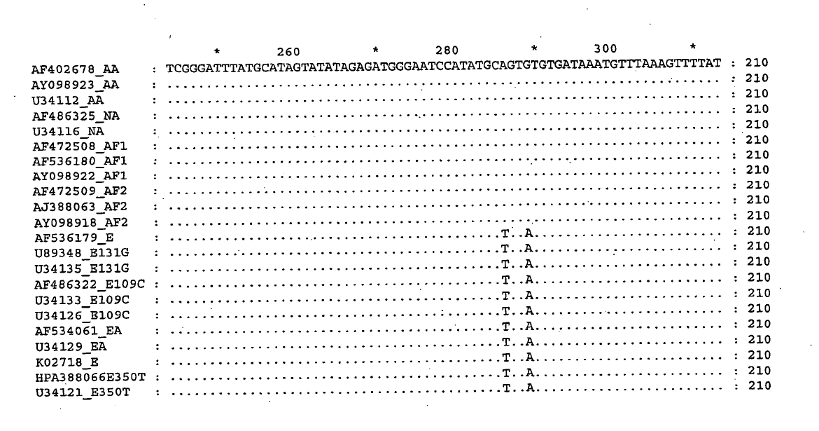

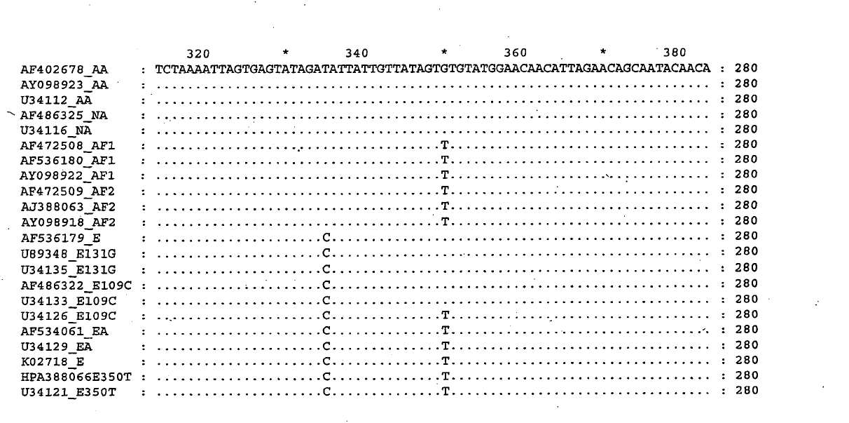

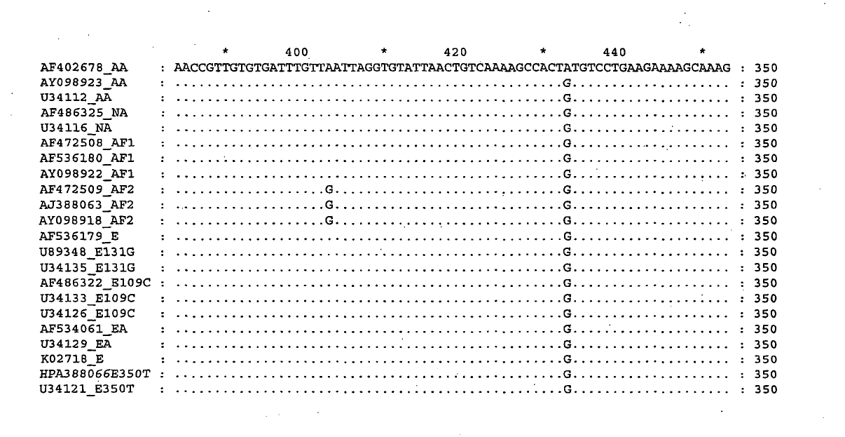

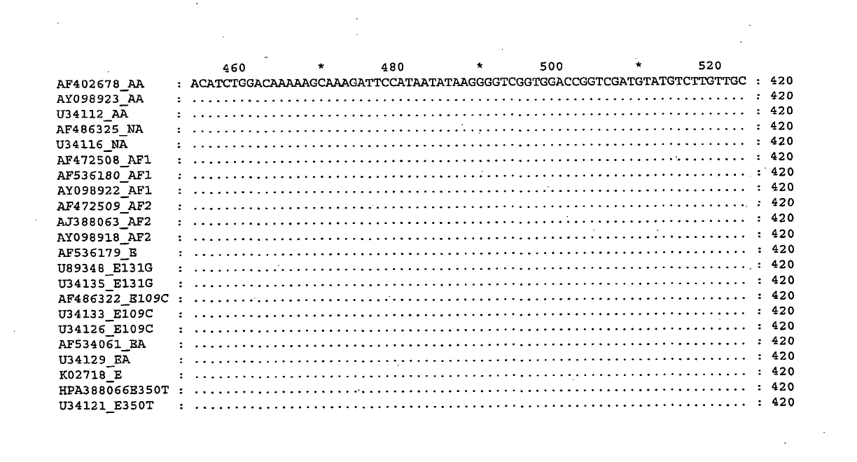

These probes can be used in combination with other probes to provide further information about HPV 16 subtypes. Examples of how these probes might be used to provide such information is provided in FIGS. 2, 4 and 5.

Table 3 contain a list of probes specifically hybridizing to BPV 16 (see FIG. 1), which may be used in combination with probes which specifically hybridise across position 143 and 145. These probes may be used together, suitably under the same conditions of hybridization, and washing. Preferred is a reverse hybridization format, such as a line probe assay format. (Preferred probes hybridizing to positions 143 and 145 have been included for completeness.)

TABLE 3

| | Sequence probe | | | Start position |
|---|---|---|---|---|
| | (key nucleotide | | Key | of probe |
| | bold and underlined) | | mutation | (reference |
| Probe name | Sequence 5′ → 3′ | Orientation | covered | K02718) |
| E6_EU_TOT_01 | gttaccaCaGttatgcac | sense | 143/145 | 136 |
| E6_EUg131_01 | gcgacccGgaaagtta | Sense | 131 | 124 |
| E6_EUg350_01 | ttgttatagtGtgtatggaac | Sense | 350 | 340 |
| E6_EUt350_02 | tgttatagtTtgtatggaaca | Sense | 350 | 341 |
| E6_EUc335_01 | gtgagtatagaCattattgtt | Sense | 335 | 324 |
| E6_EUt335_02 | tagtgagtatagaTattattgtt | Sense | 335 | 322 |
| E6a286g289_1 | ccatatgcAgtGtgtgat | Sense | 286/289 | 278 |
| E6t286a289_1 | ccatatgcTgtAtgtgat | Sense | 289/289 | 278 |
| E6EAsg178_03 | tattatCtcatgtatagttgtgg | Antisense | 178 | 164-185 |
| E6_AF_TOT_01 | gttaccaGaTttatgcac | Sense | 143/145 | 136 |
| E6AF1c132_03 | gcgacccaCaaagttac | Sense | 132 | 124 |
| E6AF2g403_03 | tgtgatttgttGattaggt | Sense | 403 | 392 |
| E6_NA_AA_01 | gttaccaCaTttatgcac | Sense | 143/145 | 136 |
| E6_AAg532_11 | tgttctCgatgatctgca | Antisense | 532 | 521-539 |

The present invention relates to all probes listed herein individually per se, and use of said probes in the detection and/or identification of HPV 16 subtypes present in a sample.

Also claimed is a probe which is the reverse complement of the above sequence. For the avoidance of doubt reverse complements of all sequences listed herein form part of the invention.

Probes for use in the present invention may have an additional spacer sequence which does not form part of the probe itself but which can allow for attachment to a solid support, for example. The spacer region may be added enzymatically or chemically and may be 5′ or 3′ of the probe.

Probes are preferably optimized for hybridization in a microtiter plate, e.g. according to the DEIA technique, or use in reverse hybridization such as a line probe assay.

### Amplification and Primers

Any HPV nucleic acid present in the sample may optionally first be amplified, for example by PCR or other suitable amplification process, prior to hybridization. Amplification of any target nucleic acid may be carried out using so called “broad spectrum” primers or primer sets that allow for amplification of all HPV 16 nucleic acid in a sample, regardless of subtype/variant.

Reference to HPV nucleic acid present in a sample thus includes nucleic acid that has been amplified from a sample, where this is clear from the context (i.e. an amplification step is present prior to hybridisation).

Thus, in one embodiment the present invention relates to a method for detection and/or subtyping of any HPV 16 nucleic acid possibly present in a biological sample, the method comprising the steps of:

(i) amplification of a polynucleic acid fragment or fragments comprising nucleotides 143 and 145 of any HPV 16 DNA in the sample, and
(ii) contacting any amplified fragments from step (i) with at least one probe capable of specific hybridization across a region including position 143 and/or 145 of HPV 16,

Suitably the region to be amplified comprises nucleotides 71-640 of the HPV 16 genome, where this numbering is given by reference to the Los Alamos reference sequence, or consists of this region, or consists essentially of this region.

Suitably the region to be amplified includes no more than fragment 71-640 of the HPV 16 genome.

According to another preferred embodiment of the present invention, the 5′ end of said 5′-primer specifically hybridizing to the A region of the genome of at least one HPV type, is situated at position 71 of the genome of HPV 16, by reference to the sequence of FIG. 1.

According to another preferred embodiment of the present invention, the 5′ end of said 3′-primer specifically hybridizing to the A region of the genome of at least one HPV type, is situated at position 640 of the HPV 16 genome, by reference to the sequence of FIG. 1.

Preferred primers for amplification of nucleic acid in a sample include:

TABLE 4

| Primer | Start | Sequence |
|---|---|---|
| set 1 | position | (5′→ 3′) |
| HPV16E6F1 | 71 | AGC AGA CAT TTT ATG CAC C |
| HPV16E6R1 | 640 | GCT CAT AAC AGT AGA GAT C |
| Universal | | Each of the primer target |
| probe | | sequences in primer set 2 |
| | | (tables 5 and 6) can be used |
| | | as probe |

The invention thus relates to HPV16E6F1 and HPV16E6R1, and use of these primers in the detection and/or identification of HPV present in a biological sample.

The invention also relates to the primer combination consisting of SEQ HPV16E6F1 and HPV16E6R1 for use in the detection and/or identification of HPV present in a biological sample.

It should also be clear that in certain cases, probes may be chosen to overlap with the primers used in the amplification step. In this case, however, the region of overlap between primer and probe should not be as long as to allow by itself duplex formation under the experimental conditions used.

Amplification may use 1 primer set creating one single amplimer—eg region 71-640. Alternatively, smaller amplimers may be generated (for example, regions A, B, C and D described below). Amplimers of less than 150 nucleotides, preferably less than 140, 130, 120, 110 or 100 nucleotides are preferred. Shorter amplimers may be better able to be amplified from paraffin embedded tissue, for example.

For example, primers suitable to amplify a shorter region covering positions 143 and 145 are:

TABLE 5

| | positions | | |
|---|---|---|---|
| Primer set | reference | Sequence | |
| (2) for: | K02718 | (5′→ 3′) | Polarity |

Region A

| | | | |
|---|---|---|---|
| Forward | 105-124 | TGTTTCAGGACCCACAGGAG | + |
| Reverse 1 | 150-170 | TAGTTGTTTGCAGCTCTGTGC | − |
| Reverse 2 | 188-208 | TTGCTTGCAGTACACACATTC | − |
| Universal | | GCGACCC(A/G)(C/G/T)AA | + |
| probe | | AGTTACCA | |

Other possible amplimers that cover regions of HPV 16 useful in subtyping include:

TABLE 6

| | positions | | |
|---|---|---|---|
| Primer set | reference | Sequence | |
| (2) for: | K02718 | (5′→ 3′) | Polarity |

Region B

| | | | |
|---|---|---|---|
| Forward | 265-284 | TAGAGATGGGAATCCATATGC | + |
| Reverse | 352-373 | CTGTTCTAATGTTGTTCCATA | − |
| | | C | |
| Universal | 322-348 | TAGTGAGTATAGA(C/T)ATT | + |
| probe | | ATTGTTATAG | |

Region C

| | | | |
|---|---|---|---|
| Forward | 373 | GCAATACAACAAACCGTTGTG | + |
| Reverse | 418-437 | GACACAGTGGCTTTTGACAG | − |
| Universal | 392 | TGTGATTTGTT(A/G)ATTAG | + |
| probe | | GTGTATT | |

Region D

| | | | |
|---|---|---|---|
| Forward | 505 | TCGATGTATGTCTTGTTGCAG | + |
| Reverse | 553-573 | ATCTCCATGCATGATTACAGC | − |
| Universal | 533-553 | CTGGGTTTCTCTACGTGTTCT | − |
| probe | | | |

All primers and universal probes are claimed per se herein, and in combination.

Further, groups of probes falling within the shorter amplimers (A, B, C and D) are also claimed—for example. 2 or more, suitably all of E6_EU_TOT_{—}01, E6_EUg131_{—}01, E6EAsg178_{—}03, E6_AF_TOT_{—}01, E6AF1c132_{—}03, E6_NA_AA_{—}01 in region A, and

Two or more, suitably all of E6_EUg350_{—}01, E6_EUt350_{—}02, E6 EUc335_{—}01, E6_EUt335_{—}02, E6a286g289_{—}1, E6t286a289_{—}1 in region B

### Universal Probes

It can be useful to check for the presence of HPV 16 DNA prior to any subtyping analysis. In this case the use of universal probes which are able to recognize any HPV16 sequence may be employed. Where amplification of target is carried out prior to subtyping then universal probes are located within the amplified region. Examples of universal probes that may be used to verify the presence of HPV 16 DNA are listed in tables above.

Any primer listed above to amplify regions A, B, C or D may be used as a universal probe for primer set 1.

Universal probes may contain inosine residues as part of the nucleic acid probe sequence, which allows for some flexibility in hybridisation to target nucleic acid, and can allow hybridisation to different HPV subtypes.

Universal probes may be used to detect HPV nucleic acid using the DEIA technique, for example as explained in WO991437 and for example in Clin Diagn Virol. 1995 February; 3(2):155-64, herein incorporated by reference. This method is used for rapid and specific detection of PCR products. PCR products are generated by a primer set, of which either the forward or the reverse primer contain biotin at the 5′ end. This allows binding of the biotinylated amplimers to streptavidin-coated microtiter wells. PCR products are denatured by sodium hydroxide, which allows removal of the non-biotinylated strand.

Specific labelled oligonucleotide probes (e.g. with digoxigenin) are hybridized to the single stranded immobilized PCR product and hybrids are detected by enzyme-labelled conjugate and colorimetric methods.

### Detection of Hybridisation

After the hybridization between the probe and any target DNA, detection of the hybridization may be carried out by any suitable means. For example, the probe and/or nucleic acid may be detectably labeled. To assist in detection it is preferred that the target and/or the signal are amplified. PCR amplification of the target DNA is especially preferred.

### Methodology

The hybridisation between probe and target is preferably carried out in the presence of a solid support, although this is not obligatory. One or more of the probe and target nucleic acid may be immobilised, for example, being fixed to a bead, plate, slide or microtitre dish. Alternatively neither probe nor target may be immobilised. Hybridisation may be carried out in the context of a liquid medium.

Detection of binding maybe carried out using flow cytometry, for example using the Luminex™ flow cytometry system (see, for example, W09714028 and http://www.luminexcorp.com/).

Detection of binding may also be carried out in the context of a microarray, using for example methods as described in EP373203, EP386229, EP0804731 and EP619321 and incorporated herein by reference. Such techniques are well known to the person skilled in the art.

According to another preferred embodiment of the present invention, the aforementioned methods of detection and/or identification of HPV are characterized further in that the hybridization step involves a reverse hybridization format. This format implies that the probes are immobilized to certain locations on a solid support.

Suitably any HPV 16 nucleic acid in a sample is amplified as described above, and the amplified HPV polynucleic acids are labelled in order to enable the detection of the hybrids formed.

According to this embodiment, at least one probe, or a set of a least 2, preferably at least 3, more preferably at least 4 and most preferably at least 5 probes is used. When at least 2 probes are used, said probes are designed in such a way that they specifically hybridize to their target sequences under the same hybridization conditions and the same wash conditions.

According to an even more preferred embodiment of the present invention, the aforementioned hybridization step is performed according to a line probe assay technique. Said technique involves a reverse hybridization assay, characterized in that the oligonucleotide probes are immobilized on a solid support as parallel lines (Stuyver et al., 1993; international application WO 94/12670). The reverse hybridization format has many practical advantages as compared to other DNA techniques or hybridization formats, especially when the use of a combination of probes is preferable or unavoidable to obtain the relevant information sought.

Alternatively, detection of BPV polynucleic acids in a biological sample may be performed by use of the DNA Enzyme Immuno Assay (DEIA).

In a preferred embodiment the invention relates to a method comprising:
1 Amplification of nucleic acid from any HPV16 present in a biological sample,
2 Detection of any HPV 16 nucleic acid present in a biological sample,
3 Subtyping of the HPV 16 nucleic acid in samples in which such HPV nucleic acid has been detected by contacting such nucleic acid with at least one probe capable of specific hybridization across a region including position 143 of the HPV 16 genome, and a probe capable of specific hybridization to the HPV 16 genome across a region including position 145 of the HPV 16 genome, positions given with reference to the sequence of Figure and then analysing HPV subtype based upon the hybridisation result so obtained.

Steps 2 and 3 may be carried out simultaneously.

### Kits

The present invention also relates to kits for use in the present invention, to detect and/or identify HPV 16 subtypes.

A kit can comprise at least 2 primers suitable for amplification of nucleic acid from the genome of HPV 16, suitably capable of amplification of at least fragment 71-640 of the HPV genome, such as primers HPV16E6F1 and HPV16E6R1. Primers for the amplification of regions A, B, C and D are also preferred, as listed in tables 5 and 6.

A kit can comprise at least 2 probes capable of specific hybridization within fragment 71-640 of the HPV 16 genome, with numbering given in respect of FIG. 1.

Preferred probes are capable of allowing discrimination between different HPV 16 subtypes, with suitable probes listed in Table 1. Suitably probes are capable of hybridization with only one HPV 16 subtype under appropriate reaction conditions.

Preferred are kits comprising at least 2 probes, the probes being useful individually or in combination to allow discrimination between C and G at position 143 in the HPV 16 genome, and T and G at position 145 in the HPV 16 genome.

Preferred are kits comprising any 2 or all 3 of the following probes (5′-3′)

1 gttaccaCaGttatgcac,
2 gttaccaGaTttatgcac,
and
3 gttaccaCaTttatgcac

A kit can comprise instructions for carrying out the above methods for HPV identification and subtyping analysis, in combination with a primer and/or probe as indicated above.

A kit can also comprise at least one primer and at least one probe, as given above.

A kit can comprise a probe or primer of the present invention immobilised onto a solid support. The support can be a bead, microtitre plate or slide, for example.

The present invention also relates to diagnostic kits for detection and/or identification of HPV 16 subtypes possibly present in a biological sample, comprising the following components: (i) at least one suitable primer or at least one suitable primer pair as defined above; (ii) at least one suitable probe, preferably at least 2, more preferably at least 3, even more preferably at least 4 and most preferably at least 5 suitable probes, optionally fixed to a solid support.

Suitably the kit additionally comprises one or more of the following:

(iii) a hybridization buffer, or components necessary for the production of said buffer, or instructions to prepare said buffer;
(iv) a wash solution, or components necessary for the production of said solution, or instructions to prepare said solution;
(v) a means for detection of the hybrids formed;
(vi) a means for attaching the probe(s) to a solid support.

For the avoidance of doubt, the present invention extends beyond use of primers and probes listed herein for the analysis of HPV 16 subtypes at positions 143 and 145. All primers and probes listed herein are claimed per se. Groups of probes and primers which may be useful in subtyping HPV 16 and which do not necessarily contain probes that are capable of hybridization across position 143 and 145 are also specifically included in the present invention. Such groups of probes can suitably be used under the same hybridization conditions to identify subtypes of HPV 16.

The present invention thus also relates to a group of probes comprising any 2, 3, 4, 5, 6, 7, 8, 9, 10, 11 or 12 of the probes listed in Table 3. Suitably any group of said probes can be used under the same hybridization conditions to assess for specific hybridization of any probe in the group. Preferred is the group of all probes listed in table 3.

Groups of probes of the present invention also include:

Two or more probes, suitably 3, 4, 5 or suitably all probes, from the list of E6_EU_TOT01, E6_EUg131_{—}01, E6EAsg178_{—}03, E6_AF_TOT_{—}01, E6AF1c132_{—}03, E6_NA_AA_{—}01;

Two or more probes, suitably 3, 4, 5 or suitably all probes, from the list of E6 EUg350_{—}1, E6_EUt350_{—}02. E6 EUc335_{—}01, E6_EUt335_{—}02, E6a286g289_{—}1, E6t286a289_{—}1.

The invention also extends to cover any primer listed in table 4, 5 or 6, and pairs of primers useful in amplification of region 71-640 of HPV 16 (given by reference to FIG. 1), or smaller fragments within said region suitably region A, B, C and D.

Isolated HPV DNA fragments 71-640, 105-170, 105-208, 265-373, 373-437 and 505-573 are also claimed, and use thereof in subtyping HPV 16.

The invention also relates to the reverse complement of all sequences.

The following definitions and explanations will permit a better understanding of the present invention.

HPV isolates that display a sequence difference of more than 10% to any previously known type in a 291 bp fragment from the L1 region (Chan et al., 1995) are classified as different HPV “types”. HPV isolates that differ between 2 and 10% are classified as different “subtypes”. If the sequence variation is below 2%, the isolates are classified within the same subtype as different “variants”. The term “type” when applied to HPV refers to any of the three categories defined above.

The target material in the samples to be analyzed may either be DNA or RNA, e.g. genomic DNA, messenger RNA, viral RNA or amplified versions thereof. These molecules are in this application also termed “nucleic acids” or “polynucleic acids”.

Well-known extraction and purification procedures are available for the isolation of RNA or DNA from a sample (e.g. in Sambrook et al., 1989).

The term “probe” according to the present invention generally refers to a single-stranded oligonucleotide which is designed to specifically hybridize to HPV polynucleic acids.

The term “primer” generally refers to a single stranded oligonucleotide sequence capable of acting as a point of initiation for synthesis of a primer extension product which is complementary to the nucleic acid strand to be copied. The length and the sequence of the primer must be such that they allow to prime the synthesis of the extension products.

Preferably the primer is about 10-50 nucleotides long. Specific length and sequence will depend on the complexity of the required DNA or RNA targets, as well as on the conditions at which the primer is used, such as temperature and ionic strength.

The expression “primer pair” or “suitable primer pair” in this invention refers to a pair of primers allowing the amplification of part or all of the HPV polynucleic acid fragment for which probes are able to bind.

The term “target” or “target sequence” of a probe or a primer according to the present invention is a sequence within the HPV polynucleic acids to which the probe or the primer is completely complementary or partially complementary (where partially complementary allows for some degree of mismatch). It is to be understood that the complement of said target sequence is also a suitable target sequence in some cases. Probes of the present invention are suitably complementary to at least the central part of their target sequence. In most cases the probes are completely complementary to their target sequence. The term “type-specific target sequence” refers to a target sequence within the polynucleic acids of a given HPV type that contains at least one nucleotide difference as compared to any other HPV-type.

“Specific hybridization” of a probe to a region of the HPV polynucleic acids means that said probe forms a duplex with part of this region or with the entire region under the experimental conditions used, and that under those conditions said probe does not form a duplex with other regions of the polynucleic acids present in the sample to be analysed. It should be understood that probes that are designed for specific hybridisation within a region of HPV polynucleic acid may fall entirely within said region or may to a large extent overlap with said region (i.e. form a duplex with nucleotides outside as well as within said region).

Suitably the specific hybridisation of a probe to a nucleic acid target region occurs under stringent hybridisation conditions, such as 3×SSC, 0.1% SDS, at 50° C.

The skilled person knows how to vary the parameters of temperature, probe length and salt concentration such that specific hybridisation can be achieved. Hybridization and Wash conditions are well known and exemplified in Sambrook, et al., Molecular Cloning: A Laboratory Manual, Second Edition, Cold Spring Harbor, N.Y., (1989), particularly Chapter II therein. When needed, slight modifications of the probes in length or in sequence can be carried out to maintain the specificity and sensitivity required under the given circumstances. Probes and/or primers listed herein may be extended by 1, 2, 3, 4 or 5 nucleotides, for example, in either direction.

Preferred stringent conditions are suitably those which allow for a type specific probe binding to only one HPV type. Thus in an embodiment of the invention the method for typing of any HPV nucleic acid possibly present in a biological sample comprises the steps of contacting any such nucleic acid with at least one probe which is capable of hybridisation to the D and/or B region of HPV under stringent conditions.

Probes which specifically hybridise to the HPV genome as defined herein suitably at least 95% complementary to the target sequence over their length, suitably greater than 95% identical such as 96%, 97%, 98%, 99% and most preferably 100% complementary over their length to the target HPV sequence. The probes of the invention can be complementary to their target sequence at all nucleotide positions, with 1, 2, or more mismatches possibly tolerated depending upon the length of probe, temperature, reaction conditions and requirements of the assay, for example.

Suitably each nucleotide of the probe can form a hydrogen bond with its counterpart target nucleotide.

Preferably the complementarity of probe with target is assessed by the degree of A:T and C:G base pairing, such that an adenine nucleotide pairs with a thymine, and such that a guanine nucleotide pairs with a cytosine, or vice versa. In the RNA form, T may be replaced by U (uracil).

Where inosine is used in universal probes, for example, then complementarity may also be assessed by the degree of inosine (probe)-target nucleotide interactions.

“Specific hybridization” of a primer to a region of the HPV polynucleic acids means that, during the amplification step, said primer forms a duplex with part of this region or with the entire region under the experimental conditions used, and that under those conditions said primer does not form a duplex with other regions of the polynucleic acids present in the sample to be analysed. It should be understood that primers that are designed for specific hybridization to a region of HPV polynucleic acids, may fall within said region or may to a large extent overlap with said region (i.e. form a duplex with nucleotides outside as well as within said region).

An embodiment of the present invention requires the detection of single base pair mismatches and stringent conditions for hybridization of probes are preferred, allowing only hybridization of exactly complementary sequences. However, it should be noted that, since the central part of the probe is essential for its hybridization characteristics, possible deviations of the probe sequence versus the target sequence may be allowable towards the extremities of the probe when longer probe sequences are used. Variations are possible in the length of the probes.

Said deviations and variations, which may be conceived from the common knowledge in the art, should however always be evaluated experimentally, in order to check if they result in equivalent hybridization characteristics as the exactly complementary probes.

Preferably, the probes of the invention are about 5 to 50 nucleotides long, more preferably from about 10 to 25 nucleotides. Particularly preferred lengths of probes include 5, 6, 7, 8, 9, 10, 11, 12, 13, 14, 15, 16, 17, 18, 19, 20, 21, 22, 23, 24 or 25 nucleotides (without counting any spacer sequences that may be present). The nucleotides as used in the present invention may be ribonucleotides, deoxyribonucleotides and modified nucleotides such as inosine or nucleotides containing modified groups which do not essentially alter their hybridization characteristics.

Probe sequences are represented throughout the specification as single stranded DNA oligonucleotides from the 5′ to the 3′ end. It is obvious to the person skilled in the art that any of the below-specified probes can be used as such, or in their complementary form, or in their RNA form (wherein T is replaced by U).

The probes according to the invention can be prepared by cloning of recombinant plasmids containing inserts including the corresponding nucleotide sequences, if need be by excision of the latter from the cloned plasmids by use of the adequate nucleases and recovering them, e.g. by fractionation according to molecular weight. The probes according to the present invention can also be synthesized chemically, for instance by the conventional phospho-triester method.

The fact that amplification primers do not have to match exactly with the corresponding target sequence in the template to warrant proper amplification is amply documented in the literature (Kwok et al., 1990). However, when the primers are not completely complementary to their target sequence, it should be taken into account that the amplified fragments will have the sequence of the primers and not of the target sequence.

Primers may be labelled with a label of choice (e.g. biotin). The amplification method used can be either polymerase chain reaction (PCR; Saiki et al., 1988), ligase chain reaction (LCR; Landgren et al., 1988; Wu & Wallace, 1989; Barany, 1991), nucleic acid sequence-based amplification (NASBA; Guatelli et al., 1990; Compton, 1991), transcription-based amplification system (TAS; Kwoh et al., 1989), strand displacement amplification (SDA; Walker et al., 1992) or amplification by means of QB replicase (Lomeli et al., 1989) or any other suitable method to amplify nucleic acid molecules known in the art.

The oligonucleotides used as primers or probes may also comprise nucleotide analogues such as phosphorothiates (Matsukura et al., 1987), alkylphosphorothiates or peptide nucleic acids (Egholm M, Buchardt O, Christensen L, Behrens C, Freier S M, Driver D A, Berg R H, Kim S K, Norden B, Nielsen P E. PNA hybridizes to complementary oligonucleotides obeying the Watson-Crick hydrogen-bonding rules. Nature. 1993 Oct. 7; 365(6446):566-8) or may contain intercalating agents (Asseline et al., 1984). As most other variations or modifications introduced into the original DNA sequences of the invention these variations will necessitate adaptions with respect to the conditions under which the oligonucleotide should be used to obtain the required specificity and sensitivity. However the eventual results of hybridization will be essentially the same as those obtained with the unmodified oligonucleotides. The introduction of these modifications may be advantageous in order to positively influence characteristics such as hybridization kinetics, reversibility of the hybrid-formation, biological stability of the oligonucleotide molecules, etc.

The term “solid support” can refer to any substrate to which an oligonucleotide probe can be coupled, provided that it retains its hybridization characteristics and provided that the background level of hybridization remains low. Usually the solid substrate will be a microtiter plate (e.g. in the DEIA technique), a membrane (e.g. nylon or nitrocellulose) or a microsphere (bead) or a chip. Prior to application to the membrane or fixation it may be convenient to modify the nucleic acid probe in order to facilitate fixation or improve the hybridization efficiency. Such modifications may encompass homopolymer tailing, coupling with different reactive groups such as aliphatic groups, NH2 groups, SH groups, carboxylic groups, or coupling with biotin, haptens or proteins.

As discussed above, hybridisation may take place in a liquid media, and binding of probe to target assessed by, for example, flow cytometry.

The term “labelled” generally refers to the use of labelled nucleic acids. Labelling may be carried out by the use of labelled nucleotides incorporated during the polymerase step of the amplification such as illustrated by Saiki et al. (1988) or Bej et al. (1990) or labelled primers, or by any other method known to the person skilled in the art. The nature of the label may be isotopic (“P, “S, etc.) or non-isotopic (biotin, digoxigenin, etc.).

The “sample” may be any material which may contain HPV nucleic acid, such as biological material, for example taken either directly from a human being (or animal), or after culturing (enrichment), or may be recombinant HPV nucleic acid expressed in a host cell. Biological material may be e.g. urine, or scrapes/biopsies from the urogenital tract or any part of the human or animal body.

The sets of probes of the present invention will generally include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more probes.

Said probes may be applied in two or more (possibly as many as there are probes) distinct and known positions on a solid substrate. Often it is preferable to apply two or more probes together in one and the same position of said solid support. The invention relates to a solid support attached to 1 or more probes of the present invention.

For designing probes with desired characteristics, the following useful guidelines known to the person skilled in the art can be applied.

Because the extent and specificity of hybridization reactions such as those described herein are affected by a number of factors, manipulation of one or more of those factors will determine the exact sensitivity and specificity of a particular probe, whether perfectly complementary to its target or not. The importance and effect of various assay conditions are explained further herein.

The stability of the [probe: target] nucleic acid hybrid should be chosen to be compatible with the assay conditions. This may be accomplished by avoiding long AT-rich sequences, by terminating the hybrids with G:C base pairs, and by designing the probe with an appropriate Tm. The beginning and end points of the probe should be chosen so that the length and % GC result in a Tm about 2° C. higher than the temperature at which the final assay will be performed. The base composition of the probe is significant because G-C base pairs exhibit greater thermal stability as compared to A-T base pairs due to additional hydrogen bonding. Thus, hybridization involving complementary nucleic acids of higher G-C content will be more stable at higher temperatures.

Conditions such as ionic strength and incubation temperature under which a probe will be used should also be taken into account when designing a probe. It is known that the degree of hybridization will increase as the ionic strength of the reaction mixture increases, and that the thermal stability of the hybrids will increase with increasing ionic strength. On the other hand, chemical reagents, such as formamide, urea, DMSO and alcohols, which disrupt hydrogen bonds, will increase the stringency of hybridization. Destabilization of the hydrogen bonds by such reagents can greatly reduce the Tm. In general, optimal hybridization for synthetic oligonucleotide probes of about 10-50 bases in length occurs approximately 5° C. below the melting temperature for a given duplex. Incubation at temperatures below the optimum may allow mismatched base sequences to hybridize and can therefore result in reduced specificity.

It is desirable to have probes which hybridize only under conditions of high stringency. Under high stringency conditions only highly complementary nucleic acid hybrids will form; hybrids without a sufficient degree of complementarity will not form. Accordingly, the stringency of the assay conditions determines the amount of complementarity needed between two nucleic acid strands forming a hybrid. The degree of stringency is chosen such as to maximize the difference in stability between the hybrid formed with the target and the nontarget nucleic acid. In the present case, single base pair changes need to be detected, which requires conditions of very high stringency.

The length of the probe sequence can also be important. In some cases, there may be several sequences from a particular region, varying in location and length, which will yield probes with the desired hybridization characteristics. In other cases, one sequence may be significantly better than another which differs merely by a single base. While it is possible for nucleic acids that are not perfectly complementary to hybridize, the longest stretch of perfectly complementary base sequence will normally primarily determine hybrid stability.

While oligonucleotide probes of different lengths and base composition may be used, preferred oligonucleotide probes of this invention are between about 5 to 50 (more particularly 10-25) bases in length and have a sufficient stretch in the sequence which is perfectly complementary to the target nucleic acid sequence.

Regions in the target DNA or RNA which are known to form strong internal structures inhibitory to hybridization are less preferred. Likewise, probes with extensive self-complementarity should be avoided. As explained above, hybridization is the association of two single strands of complementary nucleic acids to form a hydrogen bonded double strand.

It is implicit that if one of the two strands is wholly or partially involved in a hybrid that it will be less able to participate in formation of a new hybrid. There can be intramolecular and intermolecular hybrids formed within the molecules of one type of probe if there is sufficient self complementarity. Such structures can be avoided through careful probe design. By designing a probe so that a substantial portion of the sequence of interest is single stranded, the rate and extent of hybridization may be greatly increased. Computer programs are available to search for this type of interaction. However, in certain instances, it may not be possible to avoid this type of interaction.

In order to identify different HPV types with the selected set of oligonucleotide probes, any hybridization method known in the art can be used (conventional dot-blot, Southern blot, sandwich, etc.). However, in order to obtain fast and easy results if a multitude of probes are involved, a reverse hybridization format may be most convenient. In a preferred embodiment the selected probes are immobilized to a solid support in known distinct locations (dots, lines or other figures). In another preferred embodiment the selected set of probes are immobilized to a membrane strip in a line fashion. Said probes may be immobilized individually or as mixtures to delineated locations on the solid support. A specific and very user-friendly embodiment of the above-mentioned preferential method is disclosed in Example 4 of WO9914377, which may be adapted in the present invention. The HPV polynucleic acids can be labelled with biotin, and the hybrid can then, via a biotine-streptavidine coupling, be detected with a non-radioactive colour developing system.

The term “hybridization buffer” means a buffer allowing a hybridization reaction between the probes and the polynucleic acids present in the sample, or the amplified products, under the appropriate stringency conditions.

The term “wash solution” means a solution enabling washing of the hybrids formed under the appropriate stringency conditions.

Throughout this specification and the claims which follow, unless the context requires otherwise, the word “comprise”, and variations such as “comprises” and “comprising”, will be understood to imply the inclusion of a stated integer or step or group of stated integers or steps but not to the exclusion of any other integer or step or group of integers or steps. ‘Comprising’ also implies the inclusion of the meanings, ‘consisting of’ and ‘consisting essentially of’.

Preferred embodiments of the invention include:
A A method for the subtyping of any HPV 16 nucleic acid possibly present in a sample, the method comprising the steps of contacting such nucleic acid with a probe capable of specific hybridization to the HPV 16 genome across a region including position 143 of the HPV 16 genome, and a probe capable of specific hybridization to the HPV 16 genome across a region including position 145 of the HPV 16 genome, positions given with reference to the nucleotide sequence of FIG. 1, wherein the hybridization or absence of hybridization gives information as to the nucleotide at position 143 and 145 of the HPV 16 genome.
B A method according to statement A wherein the probe is capable of hybridization across a region including both positions 143 and 145.
C A method according to statement A or B wherein the probe is any one of (5′-3′):

| | |
|---|---|
| SEQ ID NO 1 | GTTACCACAGTTATGCAC |
| SEQ ID NO 2 | GTTACCAGATTTATGCAC |
| SEQ ID NO 3 | GTTACCACATTTATGCAC |

D A method according to any preceding statement wherein a set of at least 2 different hybridization probes is used simultaneously
E A method according to any preceding statement wherein three probes are used simultaneously, each probe being capable of specific hybridization across positions 143 and 145 of the HPV 16 genome, the probes comprising the following nucleotides at HPV 16 nucleotide position 143 and 145:

| | |
|---|---|
| probe 1: | C at position 143, G at position 145; |
| probe 2: | G at position 143, T at position 145; |
| probe 3: | C at position 143, T at position 145; |

or wherein the probes comprise the complementary DNA nucleotide at these positions.
F A method according to statement E wherein the three probes comprise the nucleotide sequences below (5′-3′), or the reverse complement thereof:

| | |
|---|---|
| SEQ ID NO 1 | GTTACCACAGTTATGCAC |
| SEQ ID NO 2 | GTTACCAGATTTATGCAC |
| SEQ ID NO 3 | GTTACCACATTTATGCAC |

G A method according any preceding statement comprising a probe selected from the list consisting of the following sequences (given as 5′-3′) or the reverse complement thereof:

GCGACCCGGAAAGTTA,
TTGTTATAGTGTGTATGGAAC,
TGTTATAGTTTGTATGGAACA,
GTGAGTATAGACATTATTGTT,
TAGTGAGTATAGATATTATTGTT,
CCATATGCAGTGTGTGAT,
CCATATGCTGTATGTGAT,
TATTATCTCATGTATAGTTGTGG,
GCGACCCACAAAGTTAC,
TGTGATTTGTTGATTAGGT
AND
TGTTCTCGATGATCTGCA

H A method according to any preceding statement wherein any HPV 16 nucleic acid present in the sample is first amplified, prior to hybridization.
I A method according to any preceding statement wherein the presence of HPV 16 nucleic acid is confirmed in the sample prior to the subtyping step.
J A method according to any preceding statement wherein the hybridisation between probe and target is carried out in the presence of a solid support.
K A method according to any preceding statement wherein the hybridization step is a reverse hybridization step.
L A kit comprising at least 2 primers suitable for amplification of nucleic acid containing nucleotides from the region 143-145 of HPV 16 genome, said region being defined with reference to FIG. 1.
M A kit according to statement L wherein the primers are sequence (5′→3′)

AGC AGA CAT TTT ATG CAC C,
and
GCT CAT AAC AGT AGA GAT C;

N A kit comprising at least 2 probes, said being useful individually or in combination to allow discrimination between C and G at position 143 in the HPV 16 genome, and T and G at position 145 in the HPV 16 genome.
O A kit according to statement N comprising any 2 or all 3 of (5′-3)

GTTACCACAGTTATGCAC,
GTTACCAGATTTATGCAC,
and
GTTACCACATTTATGCAC,

or the reverse complement thereof.
P A kit according to any of claims 12-15 additionally comprising any probe selected from the list consisting of (5′-3′:

GCGACCCGGAAAGTTA,
TTGTTATAGTGTGTATGGAAC,
TGTTATAGTTTGTATGGAACA,
GTGAGTATAGACATTATTGTT,
TAGTGAGTATAGATATTATTGTT,
CCATATGCAGTGTGTGAT,
CCATATGCTGTATGTGAT,
TATTATCTCATGTATAGTTGTGG,
GCGACCCACAAAGTTAC,
TGTGATTTGTTGATTAGGT,
and
TGTTCTCGATGATCTGCA;

or the reverse complement thereof.
Q A kit comprising a primer according to table 4, 5 or 6 or probe according to table 2 and instructions for carrying out the above methods for HPV subtyping.
R A kit comprising a probe capable of specific hybridization across a region including position 143 of the HPV 16 genome, or a probe capable of specific hybridization to the HPV 16 genome across a region including position 145 attached to a solid support.
S A probe set comprising 2 or more probes selected from the following list (5′-3′):

GCGACCCGGAAAGTTA,
TTGTTATAGTGTGTATGGAAC,
TGTTATAGTTTGTATGGAACA,
GTGAGTATAGACATTATTGTT,
TAGTGAGTATAGATATTATTGTT,
CCATATGCAGTGTGTGAT,
CCATATGCTGTATGTGAT,
TATTATCTCATGTATAGTTGTGG,
GCGACCCACAAAGTTAC,
TGTGATTTGTTGATTAGGT,
TGTTCTCGATGATCTGCA,
GTTACCACAGTTATGCAC
GTTACCAGATTTATGCAC,
and
GTTACCACATTTATGCAC

T A probe set according to claim 15 comprising at least one of (5′-3):

GTTACCACAGTTATGCAC,
GTTACCAGATTTATGCAC
and
GTTACCACATTTATGCAC.

### REFERENCES

Baay, M. F. D., W. G. V. Quint, J. Koudstaal, H. Hollema, J. M. Duk, M. P. M. Burger, E. Stolz, and P. Herbrink. 1995. Comprehensive study of several general and type-specific primer pairs for detection of human papillornavirus DNA by PCR in paraffin-embedded cervical carcinomas. 34:745-747.
Claas, B. C. J., W. J. G. Melchers, H. C. van der Linden, J. Lindeman, and W. G. V. Quint. 1989. Human papillomavirus detection in parafinn embedded cervical carcinomas and metastases of the carcinomas by the polymerase chain reaction. Am. J. Pathol. 135:703709.

Cornelissen, M. T. E., J. G. van den Tweel, A. P. H. B. Struyk, M. F. Jebbink, M. Bri&, J. van der Noordaa, and J. ter schegget. 1989. Localization of human pappilomavirus type 16 DNA using the polymerase chain reaction in the cervix uteri of women with cervical intraepithelial neoplasia. J. Gen. Virol. 70:2555-2562.
Cox, J. Th., A. T. Lorincz, M. H. Schiffman, M. E. Sherman, A. Cullen, and R. J. Kurman. Human papillomavirus testing by hybrid capture appears to be useful in triaging women with a cytological diagnosis of atypical squamous cells of undetermined significance. Am. J. Obstet Gynecol 1995; 164: 1461-1471.

Falcinelli, C., E. Claas, B. Kleter, and W. G. V. Quint. 1992. Detection of the human papillomavirus type 16 mRNA-transcripts in cytological abnormal scrappings. J. Med. Virol. 37:93-98.
Manos, M. M., Y. Ting, D. K Wright, A. J. Lewis, T. R. Broker, and S. M. Wolinsky. 1989. The use of polymerase chain reaction amplification for the detection of genital human papillomaviruses. Cancer Cells 7:209-214.
Saiki, R. K., D. H. Gelfland, S. Stoffel, S. J. Scharf, R. Higuchi, G. T. Horn, K B. Mullis, and H. A. Erlich. 1988. Primer-directed enzymatic amplification of DNA with a thermostable DNA polymerase. Science 239:487-491.
Sambrook et al. 1989 Molecular Cloning: A Laboratory Manual, 2nd Edition, Cold Spring Harbour Laboratory Press
Stuyver, L., R. Rossau, A. Wyscur, M. Duhamel, B. Vanderborght, H. Van Heuverswyn, and G. Maertens. 1993. Typing of hepatitis C virus isolates and characterization of new subtypes using a line probe assay. J. Gen. Virol. 74:1093-1102.
Tieben L. M., J. ter Schegget, R. P. Minnaar, J. N. Bouwes Bavinck, R I M. Berkhout, B. J. Vermeer, M. f. Jebbink, and H. L. Smits. 1993. Detection of cutaneous and genital HPV types in clinical samples by PCR using consensus primers. J. Virol. Methods 42:265-280.
Van den Brule, A. J. C., P. J. F. Snijders, R. L. J. Gordijn, O. P. Bleker, C. J. L. M. Meijer, and J. M. M. Walboomers. 1990. General primer-mediated polymerase chain reaction permits the detection of sequenced and still unsequenced human papillornavirus genotypes in cervical scrapes andcarcinomas. Int. J. Cancer 45:644-649.
Young, L. S., I. S. Bevan, M. A. Johnson, P. I. Blomfield, T. Brornidge, N. J. Maitland, and G. B. J. Woodman. 1989. The polymerase chain reaction: A new epidemiological tool for investigating cervical human papillomavirus infection. Brit. Med. J. 298:14-18.
Woodworth, C. D., Waggoner, S., Barnes, W., Stoler, M. H., Di Paolo, J. A. 1990. Human cervical and foreskin eptithelial cells immortalized by human papillomavirus DNAs exhibit displastic differentiation in vivo. Cancer Res. 50: 3709-3715.
Kwok S, Kellogg D E, McKinney N, Spasic D, Goda L, Levenson C, Sninsky J J Effects of primer-template mismatches on the polymerase chain reaction: human immunodeficiency virus type I model studies. Nucleic Acids Res. 1990, 25; 18(4):999-1005
Saiki R K, Gelfand D H, Stoffel S, Scharf S J, Higuchi R, Horn G T, Mullis K B, Erlich H A Primer-directed enzymatic amplification of DNA with a thermostable DNA polymerase. Science 1988, 29; 239(4839):487-491
Wu D Y, Wallace R B
The ligation amplification reaction (LAR)—amplification of specific DNA sequences using sequential rounds of template-dependent ligation
Genomics 1989, 4(4):560-569
Barany F
The ligase chain reaction on a PCR world.
PCR Methods Appl. 1991, 1(1):5-16
Erratum in: PCR Methods Appl. 1991, 1(2):149
Guatelli J C, Whitfield K M, Kwoh D Y, Barringer K J, Richman D D, Gingeras T R. Isothermal, in vitro amplification of nucleic acids by a multienzyme reaction modelled after retroviral replication.
Proc Natl Acad Sci USA 1990, 87(5):1874-1878
Erratum in: Proc Natl Acad Sci USA 1990 87(19):7797
Compton J
Nucleic acid sequence-based amplification.
Nature 1991, 7; 350(6313):91-92
Kwoh D Y, David G R, Whitfield K M, Chappelle H L, DiMichele L J, Gingeras T R Transcription-based amplification system and detection of amplified human immunodeficiency virus type I with a bead-based sandwich hybridization format. Proc Natl Acad Sci USA 1989, 86(4):1173-1177
G T, Fraiser M S, Schram J L, Little M C, Nadeau J G, Malinoswi D P
Strand displacement amplification—an isothermal, in vitro DNA amplification technique. Nucleic Acids Res. 1992 11; 20(7):1691-1696
Lomeli H, Tyagi S, Pritchard C G, Lizardi P M, Kramer F R
Quantitative assays based on the use of replicatable hybridization probes.
Clin Chem 1989, 35(9):1826-1831
Matsukura M, Shinozuka K, Zon G, Mitsuya H, Reitz M, Cohen J S, Broder S Phosphorothioate analogs of oligodeoxynucleotides: inhibitors of replication and cytopathic effects of human immunodeficiency virus.
Proc Natl Acad Sci USA 1987, 84(21):7706-7710
Asseline U, Delarue M, Lancelot G, Toulme F, Thuong N T, Montenay-Garestier T,
Helene C
Nucleic acid-binding molecules with high affinity and base sequence specificity: intercalating agents covalently linked to oligodeoxynucleotides.
Proc Nad Acad Sci USA 1984, 81(11):3297-3301
Bej A K, Steffan R J, DiCesare J, Haff L, Atlas R M
Detection of coliform bacteria in water by polymerase chain reaction and gene probes.
Appl Environ Microbiol. 1990, 56(2):307-314
Bej A K, Mahbubani M H, Miller R, DiCesare J L, Haff L, Atlas R M
Multiplex PCR amplification and immobilized capture probes for detection of bacterial pathogens and indicators in water.
Mol Cell Probes 1990, 4(5):353-365
Remmink A J, Walboomers J M, Helmerhorst T J, Voorhorst F J, Rozendaal L, Risse E K, Meiier C J, Kenemans P. persistent high-risk HPV genotypes in dysplastic cervical lesions is associated with progressive disease: natural history up to 36 months.
Int J Cancer. 1995 May 4; 61(3):306-11.
Chan S Y, Delius H, Halpern A L, Bernard H U. Analysis of genomic sequences of 95 papillomavirus types: uniting typing, phylogeny, and taxonomy.
J Virol. 1995 May; 69(5):3074-83.
de Villiers E M, Fauquet C, Broker T R Bernard H U, zur Hausen H. Classification of papillomaviruses.
Virology. 2004 Jun. 20; 324(1):17-27.
Altschul S F, Gish W, Miller W, Myers E W, Lipman D J Basic local alignment search tool.
J Mol Biol. 1990 Oct. 5; 215(3):403-10
Altschul S F, Madden T L, Schafer A A, Zhang J. Zhang Z, Miller W, Lipman DJ. Gapped BLAST and PSI-BLAST: a new generation of protein database search programs. Nucleic Acids Res. 1997 Sep. 1; 25(17):3389-402.
Zella D, Cavicchini A, Cattaneo E, Ciniarelli A, Bertazzoni U. Utilization of a DNA enzyme immunoassay for the detection of proviral DNA of human immunodeficiency virus type 1 by polymerase chain reaction. Clin Diagn Virol. 1995 February; 3(2):155-64

### EXAMPLE 1

### General

Total DNA can be extracted from cervical scrape or paraffin-embedded biopsy specimens using known techniques.

DNA from HPV 16 can be specifically amplified by the primers described in the present invention. Amplimers can be analyzed by stringent reverse hybridization to a strip, comprising subtype/variant specific probes. Hybridization patterns can be interpreted to deduce the presence of specific variants in the sample.

### DNA Isolation

DNA can be isolated from cervical scrapes by the MagNA Pure LC system.

DNA can be isolated from paraffin-embedded biopsy specimens by incubation with proteinase K.

### Amplification

BPV16 E6 sequences can be amplified by PCR, using the following conditions:

Composition of the PCR mix.

| | Volume | | | |
|---|---|---|---|---|
| Component | (μl) | | [Stock] | [final] |
| PCR buffer II | 5 | | 10 x | 1 x (=10 |
| (Applied) | | | | mM Tris- |
| | | | | HCl pH 8.3 |
| | | | | and 50 |
| | | | | mM KCl) |
| MgCl₂ (Applied) | 5 | | 25 mM | 2.0 mM |
| dNTPs (Amersham/ | 10 | | 1 mM each | 0.2 mM each |
| Pharmacia) | | | | |
| Primers | 0.1 | each | 200 pmol/μl | 0.4 μM |
| AmpliTaq Gold | 0.3 | | 5 U/μl | 1.5 U per |
| (Applied) | | | | reaction |
| DNA | X | μl | — | — |
| Water | Up to 50 | μl | | |

### PCR Profile and LiPA Profile

| | | | |
|---|---|---|---|
| activation of the AmpliTaq Gold | 9 min 94° C. | | |
| DNA polymerase: | | | |
| denaturation: | 30 sec 94° C., | | |
| annealing: | 45 sec 52° C., | {close oversize brace} | 40 cycles |
| elongation: | 45 sec 72° C. | | |
| final extension: | 5 min 72° C. | | |

### Reverse Hybridization

A PCR product can be generated, using biotinylated primers. The double-stranded PCR product can be denatured under alkaline conditions and added to the strip in a hybridisation buffer (3×SSC, 0.1% SDS). After hybridization and stringent washing at 50° C., the hybrids can be detected by addition of a streptavidin-conjugate and a substrate, generating a purple precipitate at the probe line. Hybridisation patterns can be visually interpreted.

LiPA profile

| Step | Temperature | Incubation time |
|---|---|---|
| Denaturation | RT | 10 min |
| Hybridization | 5o or 51° C. | 60 min |
| Stringent wash | 50 or 51° C. | 30 min |
| Conjugate | RT | 30 min |
| Substrate | RT | 30 min |

A detailed reference for a LIPA protocol is found in Kleter et al., J Clin Microbiol. 1999 August; 37(8):2508-17. incorporated herein by reference.

1. A method for identification of an HPV16 lineage group in a sample, the lineage groups being selected from the group consisting of:
1) European/Asian;
2) American Asian/North Amercian; and
3) African,
the method comprising the steps of:
(a) subtyping of any HPV 16 nucleic acid possibly present in a sample by contacting such nucleic acid simultaneously with three probes, each probe being capable of specific hybridization across positions 143 and 145 of a HPV 16 genome, these positions given with reference to the nucleotide sequence of FIG. 1,
wherein the hybridization or absence of hybridization of the probes in the probe set gives information as to the nucleotide at position 143 and 145 of any HPV 16 nucleic acid present in the sample,
and wherein the probes comprising the following nucleotides at HPV 16 nucleotide position 143 and 145:

| | |
|---|---|
| probe 1: | C at position 143, G at position 145; |
| probe 2: | G at position 143, T at position 145; |
| probe 3: | C at position 143, T at position 145 |

(or wherein the probes comprise the complementary DNA nucleotide at these positions); and
(b) assigning an HPV 16 lineage group to a sample using the following table based upon the hybridization pattern obtained:

| | Position 143 | Position 145 |
|---|---|---|
| European/asian | C | G |
| African | G | T |
| North | C | T |
| American/ | | |
| American-asian | | |

2. A method according to claim 1 wherein the three probes are used in the absence of any other probe from the HPV E6 region which is informative for HPV 16 subtype.

3. A method according to claim 1 wherein the three probes comprise or consist of the nucleotide sequences below (read 5′-3′), or the reverse complement thereof:

| | |
|---|---|
| SEQ ID NO 1 | GTTACCACAGTTATGCAC |
| SEQ ID NO 2 | GTTACCAGATTTATGCAC |
| SEQ ID NO 3 | GTTACCACATTTATGCAC |

4. A method according to claim 1 wherein any HPV 16 nucleic acid present in the sample is first amplified, prior to hybridization.

5. A method according to claim 1 wherein the presence of HPV16 nucleic acid is confirmed in the sample prior to the subtyping step.

6. A method according to claim 1 wherein the hybridisation between probe and target is carried out in the presence of a solid support.

7. A method according to claim 1 wherein the hybridization step is a reverse hybridization step.

8. A kit comprising at least 2 primers suitable for amplification of nucleic acid containing nucleotides from the region 143-145 of HPV 16 genome, said region being defined with reference to FIG. 1.

9. A kit according to claim 8 wherein the primers are sequence (5′→3′):
AGC AGA CAT TTT ATG CAC C,
and
GCT CAT AAC AGT AGA GAT C;

10. A kit according to claim 8 additionally comprising a universal probe from table 5 or probe from table 3 which hybridizes within a region amplified by the primers.

11. A kit comprising at least 2 probes, said probes being useful individually or in combination to allow discrimination between C and G at position 143 in the HPV 16 genome, and T and G at position 145 in the HPV 16 genome.

12. A kit according to claim 11 comprising any 2 or all 3 of (5′-3′):
GTTACCACAGTTATGCAC,
GTTACCAGATTTATGCAC,
and
GTTACCACATTTATGCAC,
or the reverse complement thereof.

13. A kit according to claim 8 further comprising any probe selected from (5′-3′):
GTTACCACAGTTATGCAC,
GCGACCCGGAAAGTTA,
TTGTTATAGTGTGTATGGAAC,
TGTTATAGTTTGTATGGAACA,
GTGAGTATAGACATTATTGTT,
TAGTGAGTATAGATATTATTGTT,
AGTGAGTATAGATATTATTGTT,
CCATATGCAGTGTGTGAT,
CCATATGCTGTATGTGAT,
TATTATCTCATGTATAGTTGTGG,
GTTACCAGATTTATGCAC,
GCGACCCACAAAGTTAC,
TGTGATTTGTTGATTAGGT,
GTTACCACATTTATGCAC,
TGTTCTCGATGATCTGCA,
TGTTTCAGGACCCACAGGAG,
TAGTTGTTTGCAGCTCTGTGC,
TTGCTTGCAGTACACACATTC,
GCGACCC(A/G)(C/G/T)AAAGTTACCA,
TAGTGAGTATAGA(C/T)ATTATTGTTATAG,
TGTGATTTGTT(A/G)ATTAGGTGTATT,
and
CTGGGTTTCTCTACGTGTTCT;
or the reverse complement thereof.

14. A kit comprising a primer according to table 4, 5 or 6 or probe according to table 2 and instructions for carrying out HPV identification and subtyping and identification of an HPV16 lineage group analysis.

15. A kit comprising a probe capable of specific hybridization across a region including position 143 of the HPV 16 genome, or a probe capable of specific hybridization to the HPV 16 genome across a region including position 145, attached to a solid support.

16. A probe set comprising 2 or more probes selected from (5′-3′):
GTTACCACAGTTATGCAC,
GCGACCCGGAAAGTTA,
TTGTTATAGTGTGTATGGAAC,
TGTTATAGTTTGTATGGAACA,
GTGAGTATAGACATTATTGTT,
TAGTGAGTATAGATATTATTGTT,
AGTGAGTATAGATATTATTGTT,
CCATATGCAGTGTGTGAT,
CCATATGCTGTATGTGAT,
TATTATCTCATGTATAGTTGTGG,
GTTACCAGATTTATGCAC,
GCGACCCACAAAGTTAC,
TGTGATTTGTTGATTAGGT,
GTTACCACATTTATGCAC,
TGTTCTCGATGATCTGCA,
TGTTTCAGGACCCACAGGAG,
TAGTTGTTTGCAGCTCTGTGC,
TTGCTTGCAGTACACACATTC,
GCGACCC(A/G)(C/G/T)AAAGTTACCA,
TAGTGAGTATAGA(C/T)ATTATTGTTATAG,
TGTGATTTGTT(A/G)ATTAGGTGTATT,
and
CTGGGTTTCTCTACGTGTTCT;
or the reverse complement thereof.

17. A probe set according to claim 15 comprising at least one of (5′-3′):
GTTACCACAGTTATGCAC,
GTTACCAGATTTATGCAC
and
GTTACCACATTTATGCAC.
